(19) United States
(12) Patent Application Publication
(10) Pub. No.: US 2021/0089812 A1
(43) Pub. Date: Mar. 25, 2021

(54) Medical Imaging Device and Image Processing Method

(71) Applicant: Hitachi, Ltd., Chiyoda-ku, Tokyo (JP)

(72) Inventors: Yun LI, Tokyo (JP); Takashi TOYOMURA, Tokyo (JP); Toshinori MAEDA, Tokyo (JP); Zisheng LI, Tokyo (JP)

(21) Appl. No.: 16/630,581

(22) PCT Filed: Jun. 7, 2018
(86) PCT No.: PCT/JP2018/021926

§ 371 Date: Jan. 13, 2020

(30) Foreign Application Priority Data
Jul. 28, 2017 (JP) 2017-146782

(51) Int. Cl. G06K 9/62; G06K 9/46; G06K 9/20; A61B 8/14; A61B 8/00; A61B 8/08

(52) U.S. Cl. CPC G06K 9/623; G06K 9/46; G06K 9/2081; G06K 2209/05; A61B 8/4444; A61B 8/5207; A61B 8/14

(57) ABSTRACT

Provided is a technique for automatically extracting a cross section with a high degree of precision and at high speed, with avoiding problems of operator dependence and imaging target dependence, from 3D volume data or temporally sequential 2D or 3D images or 3D volume data, acquired by a medical imaging device, when determining the cross section used for diagnosis and measurement. An image processor of an imaging device is provided with a cross section extractor for extracting a specified cross section from imaged data. The cross section extractor determines the specified cross section by using a learning model trained in advance to output discrimination scores for a plurality of cross sectional image data, the discrimination score representing spatial or temporal proximity to the specified cross section. The learning model is a downsized model obtained by integrating a highly trained model having a large number of layers, with an untrained model having less number of layers, followed by retraining.

### TECHNICAL FIELD

The present invention relates to a medical imaging device, including an ultrasound imaging device, an MRI device, and a CT device. More particularly, the present invention relates to techniques for selecting a specified cross section to be displayed, from a three-dimensional image, or two-dimensional (2D) time-series images or three-dimensional (3D) time-series images, being acquired by the medical imaging device.

### BACKGROUND ART

Medical imaging devices are used to acquire and then display a morphological image of a target region. In addition, the medical imaging devices can also be used to acquire morphological information and functional information quantitatively. One of examples of such usage may be measurement of estimated weight of an unborn baby (fetus) for observing growth thereof, by the use of an ultrasound imaging device. This type of measurement is performed according to a process, roughly divided into three steps; acquiring images, selecting an image for measurement (measurement image), and performing the measurement. In the step of acquiring images, a target region and its surroundings are imaged sequentially, thereby acquiring a plurality of two-dimensional cross-sectional images or volume data thereof. In the step of selecting the measurement image, a cross sectional image optimum for measurement is selected from the acquired data. In the step of performing the measurement, a head region, an abdominal region, and a leg region are measured for the case of measuring the estimated fetal weight, and calculations are performed on measured values according to a predetermined calculation formula, thereby obtaining a weight value. Measuring the head region or the abdominal region requires surface traces, and it has been time consuming. However, in recent years, there are suggested automatic measurement techniques that perform the traces automatically, followed by specific calculations (see Patent Literature 1 and other similar documents). This technique brings about workflow improvement in the measurement.

In the examination, however, the step of selecting of the measurement image after acquiring images takes the most time and effort. For the case of a fetus, in particular, it is difficult to estimate and visualize a position of a measurement cross section, within the abdomen of the fetus as the examinee, and thus it takes time to acquire the cross section. In order to solve the problem of difficulties in acquiring such cross section necessary for fetal examination, Patent Literature 2 discloses that a high echo area is extracted from three-dimensional data, and a cross section is selected on the basis of three-dimensional features of thus extracted high echo area. Specifically, in selecting the cross section, matching is performed with prepared template representing the three-dimensional features, and a cross section which matches with the template is determined as a cross section to be selected.

### CITATION LIST

### Patent Literature

### Patent Literature 1: WO2016/190256

### Patent Literature 2: WO2012/042808

### DISCLOSURE OF THE INVENTION

### Problems to be Solved by the Invention

Typically, an ultrasound image has characteristics including that image data may be different depending on an imaging operator at every imaging time (operator dependence), and that image data may be different depending on a constitutional predisposition and a disease of an imaging target (imaging target dependence). The operator dependence is caused by the following reason; that is, it is performed manually at every imaging time, to apply ultrasound waves and search a body for a region to be acquired as a cross sectional image or as volume data, and thus it is difficult to acquire completely identical data, even though an identical operator performs the examination on an identical patient. The imaging target dependence is caused by the following reason; that is, sound-wave propagation velocity and an attenuation rate within a body are different depending on the constitutional predisposition of the patient, and the shape of an organ is not perfectly identical between different patients due to the type of disease and individual variations. In other words, it is difficult to obtain an image that is ideal for measurement irrespective of which number is the imaging time and who is the patient, since there are influences of the operator dependence and the imaging target dependence.

The data thus acquired tends to include problems such as discrepancies with respect to the ideal position, an unclear image, and differences in a characteristic form.

The technique disclosed by Patent Literature 2 determines a cross section by matching with the templates prepared in advance, thus failing to address the aforementioned operator dependence and the imaging target dependence.

MRI devices or CT devices have less operator dependence relative to ultrasound imaging devices. However, it is difficult to determine a cross section by matching with a template, due to variations among individuals, or due to change in the shape of organs such as the heart and lungs in time-series images even in an identical person. In recent years, it is attempted to apply DL (Deep learning) techniques to improve an image quality or to determine a specific disease. In order to achieve discriminability with a high degree of precision in the DL technique, hardware with high processing power is required, together with long processing time. Thus it is difficult to install such technique on a conventionally used medical imaging device, or on a medical imaging device that needs high-speed processing.

In view of the situation above, an objective of the present invention is to avoid the problems of operator dependence and imaging target dependence, providing a technique for automatically extracting a cross section with high precision at high speed, when determining the cross section used for diagnosis and measurement, from 3D volume data acquired by a medical imaging device, or temporally sequential 2D or 3D images or 3D volume data.

### Means for Solving the Problems

In order to solve the problems above, the present invention provides a learning model that is trained to output as a discrimination score, spatial or temporal distance between a cross section to be extracted (target cross section) and a plurality of cross sections selected from processing target data, where the trained model is suitable for extracting the target cross section and easily implementable in a medical imaging device. Then, aptitude scores of cross sectional images of the processing target are calculated by using the model obtained by machine learning, thereby achieving extraction of an image of the target cross section with a high degree of precision.

The medical imaging device of the present invention includes an imager configured to collect image data of a subject, and an image processor configured to extract a specified cross section from the image data collected by the imager, wherein the image processor is provided with a model introducer configured to introduce a learning model being trained in advance to output discrimination scores for the image data of a plurality of cross sections, the discrimination score representing spatial or temporal proximity to the specified cross section, and a cross section extractor configured to select a plurality of cross sectional images from the image data and to extract the specified cross section on the basis of a result of applying the learning model to the cross sectional images being selected. The learning model is provided by integrating a feature extraction layer of a trained model, with a discrimination layer of an untrained model, and reduced in size. Thus, this learning model has a structure of layers simpler than the trained model prior to the integration.

An image processing method of the present invention determines from imaged data, a target cross section as a processing target and presents thus determined cross section, including a step of preparing a learning model being trained in advance to output discrimination scores for the image data of a plurality of cross sections, the discrimination score representing spatial or temporal proximity to the specified cross section, and a step of obtaining a distribution of discrimination scores of the plurality of cross sectional images selected from the imaged data, by using the learning model, and determining the target cross section on the basis of the distribution of the discrimination scores. This learning model is a downsized model obtained by integrating a feature extraction layer of a trained model that is trained in advance by using as learning data, the plurality of cross sectional images and the image of the target cross section constituting the imaged data, with a discrimination layer of an untrained model, followed by retraining.

### Advantages of the Invention

According to the present invention, the learning model is applied to extraction of the cross section, thereby achieving reduction of manual-operation dependence and also reduction of examination time, in automatic extraction of the cross sectional image optimum for measurement. In addition, the small and simple model, being downsized with keeping a high degree of precision, is employed as the precise and complex learning model. Accordingly, this allows installation of the learning model on the medical imaging device, with maintaining a standard scale of an image processor within the device, as well as achieving high-speed processing.

### BRIEF DESCRIPTION OF THE DRAWINGS

FIG. 1 illustrates an overall configuration of a medical imaging device;

FIG. 2 illustrates a configuration of essential parts of an image processor according to a first embodiment;

FIG. 3 is a flowchart showing processing steps of the image processor according to the first embodiment;

FIG. 4 is a block diagram showing a configuration of the medical imaging device (ultrasound imaging device) according to a second embodiment;

FIG. 5 illustrates integration and downsizing of learning models;

FIG. 6 illustrates integration and downsizing of learning models using CNN;

FIG. 7 illustrates a training process of the learning model;

FIG. 8 illustrates a cross-section selecting process according to the second embodiment;

FIG. 9 is a flowchart showing processing steps of cross section extraction according to the second embodiment;

FIG. 10 illustrates a search area for selecting a cross section according to the second embodiment;

FIG. 11 is a flowchart showing a process for adjusting the cross section being extracted according to the second embodiment;

FIG. 12 illustrates a display example of the extracted cross section and GUI for adjusting the cross section;

FIG. 13 illustrates measurement cross sections for measuring a fetal weight;

FIGS. 14(a) to (c) illustrate measurement positions of the measurement cross sections as shown in FIG. 13; and

FIG. 15 illustrates acquiring of 2D time-series images and generating a group of cross sections from data memory.

### BEST MODE FOR CARRYING OUT THE INVENTION

There will now be described embodiments of the present invention, with reference to the accompanying drawings.

### First Embodiment

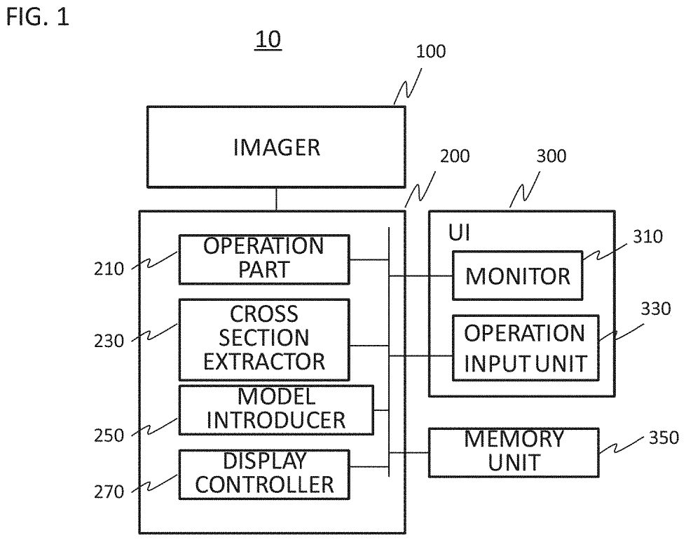

As shown FIG. 1, a medical imaging device 10 of the present embodiment is provided with an imager 100 configured to take an image of a subject and acquire image data, an image processor 200 configured to perform image processing on the image data acquired by the imager 100, a monitor 310 configured to display an image acquired by the imager 100 or an image processed by the image processor 200, and an operation input unit 330 for a user to enter commands and data necessary for the processing in the imager 100 and in the image processor 200. Typically, the monitor 310 is placed in proximity to the operation input unit 330, functioning as a user interface (UI) 300. The medical imaging device 10 may further be provided with a memory unit 350 for storing the image data obtained by the imager 100, data used in the processing by the image processor 200, and processing results thereof.

The imager 100 may be structured variously depending on modality. For the case of an MRI device, there are provided, for example, a magnetic field generation means for collecting magnetic resonance signals from the subject that is placed in a static magnetic field. For the case of a CT device, there are provided an X-ray source for applying X-rays to the subject, an X-ray detector for detecting X-rays passing through the subject, and a mechanism for rotating the X-ray source and the X-ray detector around the subject. For the case of an ultrasound imaging device, there is provided a means for transmitting ultrasound waves to the subject and receiving the ultrasound waves being reflected waves from the subject, so as to generate an ultrasound image. A method of generating image data in the imager may also be various depending on modality, but any data finally obtained may be volume data (3D image data) or 2D time-series image data or time-series volume data. Such data will be collectively referred to as “volume data” in the following description.

The image processor 200 is provided with a cross section extractor 230 configured to extract a specified cross section (referred to as “target cross section”), from the 3D volume data delivered from the imager 100, and model introducer 250 configured to introduce a learning model (discriminator) into the cross section extractor 230, the learning model inputting information of a plurality of cross sections included in the 3D volume data and outputting a score representing proximity between the cross sections and the target cross section, according to a feature of each cross section. The target cross section may be different depending on a diagnostic purpose or an objective of image processing on the cross section. In this example here, the target cross section is assumed as suitable for measuring the size (such as width, length, diameter, and circumferential length) of a structure, e.g., a specified organ and a region included in the cross section. The image processor 200 may further be provided with an operation part 210 for performing further measurement and other operations on image data of the cross section extracted by the cross section extractor 230, and a display controller 270 for displaying on the monitor 310, the cross section extracted by the cross section extractor 230 and results and other information from the operation part.

A learning model used by the cross section extractor 230 is a machine learning model that has been trained to output scores representing similarity between a correct image and a large number of cross sectional images included in the 3D volume data where the target cross section is already known, considering an image of the target cross section as the correct image, and for example, the learning model may comprise CNN (convolution neural network). A highly trained model (the first trained model) is integrated with an untrained model having less number of layers than the first trained model, and then, the learning model of the present embodiment is created as a downsized model (the second trained model). After the integration, the downsized model has already been trained in the same manner as trained CNN. The first trained model includes many layers and a large number of iterations are required for learning, but learning precision is high. The downsized model is obtained by combining a part of layers of the model trained with high precision, that is, a particularly trained layer with high precision including a feature extraction layer, for instance, with a layer of relatively low learning contribution in the untrained model, e.g., a discrimination layer within lower-level layers in CNN. Thus, the downsized model has a simple configuration with less number of layers, relative to the first trained model. Employing such downsized learning model allows installation of the learning model on the medical imaging device, with reducing processing time of the image processor 200. A specific structure and learning process of the learning model will be described in detail in the following embodiments.

The learning model (downsized model) is created in advance in the medical imaging device 10, or for instance, by a computer independent of the medical imaging device 10, and stored in the memory unit 350. Depending on variations of discrimination tasks, more than one downsized model may be stored. For example, when there is a plurality of cross sections as measurement targets, the downsized models may be created respectively for the measurement targets; e.g., the head, the chest, and the legs. When the type of target cross section is more than one, the downsized model may be created in response to the type of the target cross section. When there is a plurality of downsized models, the model introducer 250 calls a model necessary for the discrimination task, and passes the model to the cross section extractor 230.

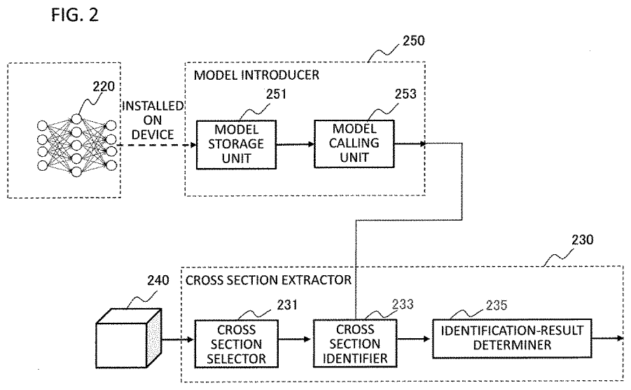

As shown in FIG. 2, the model introducer 250 is provided with a model storage unit 251 for reading the learning model 220 suitable for a processing target from the memory unit and storing the model, and a model calling unit for calling the learning model from the model storage unit 251 and applying the model to the cross section extractor 230. In addition, the cross section extractor 230 is provided with a cross section selector 231 for selecting image data of a plurality of cross sections from the volume data 240, a cross section identifier 233 for outputting scores for the image data of the cross sections selected by the cross section selector 231, the score representing proximity between the cross sections and the target cross section, by using the learning model read out from the model introducer 250, and a determiner 235 for analyzing the scores outputted from the cross section identifier 233 and determining the target cross section.

A part of or all of functions of the image processor 200 can be implemented by software that is executed by a CPU. Apart of the imager for generating image data and a part of the image processor may be implemented by hardware such as ASIC (Application Specific Integrated Circuit) and FPGA (Field Programmable Gate Array).

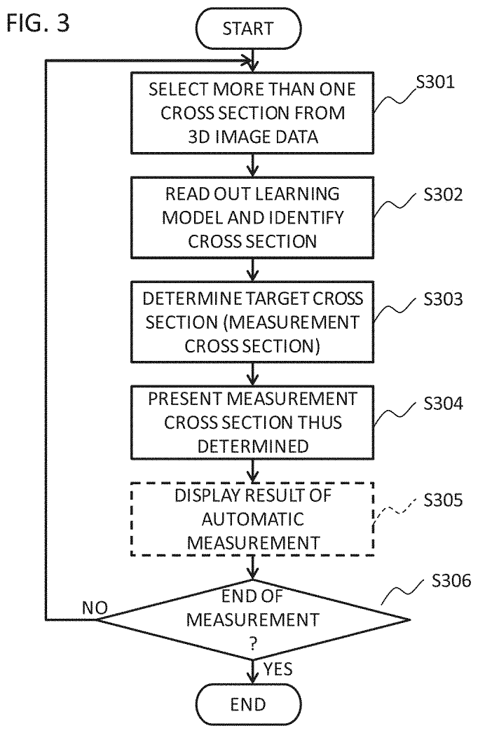

With the configuration as described above, an operation of the medical imaging device of the present embodiment, mainly processing steps of the image processor 200, will be described with reference to FIG. 3. There will be described an example where imaging and image displaying are executed in parallel.

As a precondition, a user may select a type of the target cross section via the operation input unit 330, for example. Types of the target cross section may include, a type depending on difference in purpose, for example, the cross section for measurement or the cross section for ensuring a direction where a structure extends, and a type depending on difference in measurement targets (such as a region, an organ, and a fetus). Such information may be entered at the time of setting imaging conditions, or this information may be set as a default when the imaging conditions are provided.

Upon receipt of 3D image data obtained by imaging according to the imager 100, the cross section selector 231 selects a plurality of cross sections from the 3D image data (S301). In the case where an orientation of the target cross section in the image space is known, the cross section selector selects more than one cross sections along the direction of the orientation and passes them to the cross section identifier 233. For example, when Z-axis is set as a body axis direction and the cross section is known to be parallel to the XY plane, XY planes at specific intervals are selected. Since the target cross section cannot be kept constant depending on structures (tissue or regions) included in the volume data, cross sections at various orientations may be selected in such a case. Preferably, the cross sections may be selected according to an approach, so-called, “coarse to fine approach”. In this approach, selection by the cross section selector 231 and identification by the cross section identifier 233 are repeated, and an area searched for selecting the cross sections (referred to as “search area”) is narrowed down starting from a relatively large area at each iteration. As the search area becomes narrower, intervals between the cross sections to be selected are made narrower, and further, the number of angles of the cross sections may also be increased.

On the other hand, the model introducer 250 reads out a learning model from the memory unit 350, in response to the type of the preset target cross section, and stores the learning model in the model storage unit 251. When the cross sections selected by the cross section selector 231 are passed to the cross section identifier 233, the model calling unit 252 calls from the model storage unit 251, the learning model to be applied. The cross section identifier 233 uses the learning model thus called to perform feature extraction and identification (discrimination) of the selected cross sections, and outputs a distribution of scores as a result of the identification (S302). The distribution of scores represents plotting of scores indicating a degree of similarity between the target cross section and the cross sections as processing targets, where distance values from the target cross section to the plurality of cross sections are plotted in the distribution. The distribution shows that the higher is the score, the cross section with the score is closer to the target cross section, in terms of spatial distance. The scores in the distribution have numerical values from 0 to 1 where the score of the cross section agreeing with the target cross section is set to 1.

The identification-result determiner 235 receives the distribution of scores being the result from the cross section identifier 233, and determines as the target cross section, a cross section that has the best score as a final result, i.e., the cross section having the score equal to 1 or the closest to 1, in the aforementioned example (S303).

After the target cross section is extracted by the cross section extractor 230, the display controller 250 displays this extracted cross section on the monitor 310 (S304). When the operation part 240 is provided with an automatic measurement function, the structures on the cross section are measured and the result of the measurement is displayed on the monitor 310 via the display controller 250 (S305). When there is a plurality of discrimination tasks, or reprocessing becomes necessary due to user's adjustment, the processing returns to step S301 (S306), and S301 to S304 (S305) are repeated.

According to the present embodiment, using a model (discriminator) that is trained in advance to identify a cross section being the closest to the target cross section, allows determination of the target cross section within a short time and automatically. Further according to the present embodiment, the learning model is obtained by integrating a partial layer of the model being highly trained in advance, with a partial layer of an untrained model with a relatively simple structure, and then retrained. Therefore, this learning model can be easily implemented in the imaging device and processing time using the learning model can be reduced significantly. Consequently, the time from imaging until displaying the target cross section, or until measurement using the target cross section can be reduced, and this enhances real-time characteristics.

In the first embodiment, there has been described the example where the processing target is 3D volume data. As a similar example, the present invention is also applicable to time-series data. That is, in the case where the time-series data is 2D time-series data, replacing one dimension of 3D by temporal dimension, and this 2D time-series data comprises various time-phase sectional images. When an image at a specified time phase is assumed as the target cross section, 2D time-series image data being imaged is inputted into the image processor 200 in a specified time unit, and then the aforementioned processing is performed, thereby automatically identifying the cross section in the target time phase and displaying the cross section.

If the 2D time-series image data does not include the target cross section, the processing by the image processor 200 is performed in parallel with continuous imaging, and this allows a search for the target cross section. In the case of the 2D time-series image data, it is sufficient for the cross section selector 231 to select only an imaged cross section (a plane in one direction), and this enables high-speed processing. It is further possible to select all of the imaged cross sections taken at predetermined intervals.

There has been described so far one embodiment of the present invention that is applicable irrespective of modality. Another embodiment of the present invention will be described in the following, where the present invention is applied to an ultrasound imaging device.

### Second Embodiment

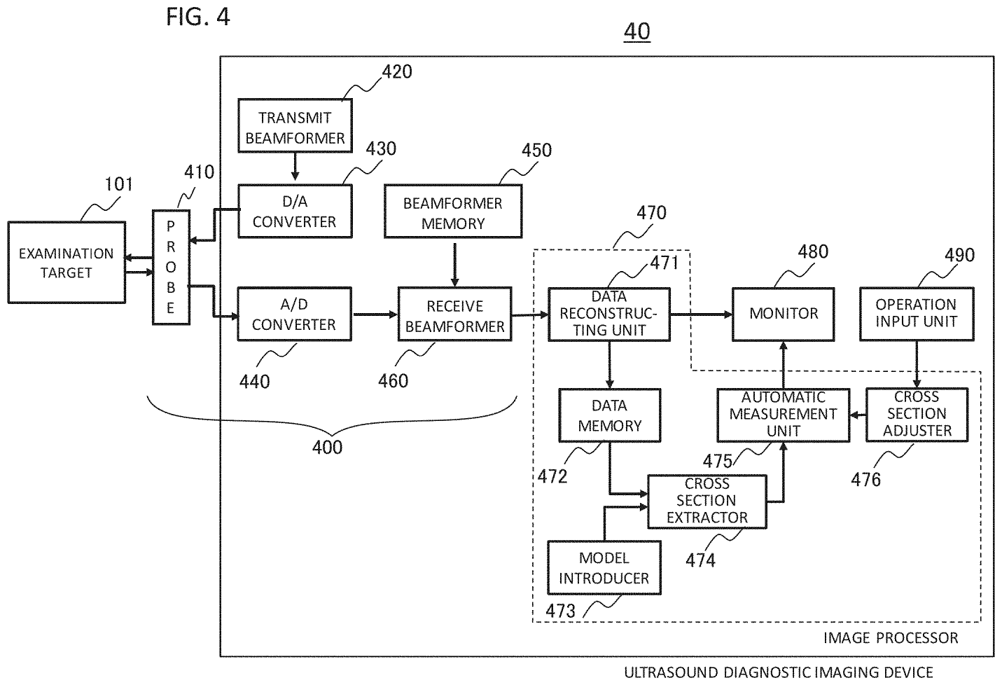

Initially, with reference to FIG. 4, there will be described a configuration of the ultrasound imaging device to which the present invention is applied. The ultrasound imaging device 40 of the present invention comprises an ultrasound imager 400 including a probe 410, a transmit beamformer 420, a D/A converter 430, an A/D converter 440, a beamformer memory 450, and a receive beamformer 460, and further comprises an image processor 470, a monitor 480, and an operation input unit 490.

The probe 410 comprises a plurality of ultrasound elements arranged along a predetermined direction. For example, each of the ultrasound elements is a ceramic element made of ceramic, for instance. The probe 410 is placed in such a manner that the probe comes into contact with the surface of the examination target 101.

The transmit beamformer 420 allows transmission of ultrasonic waves from at least a part of the plurality of ultrasound elements via the D/A converter 430. Delay time is given to each of the ultrasonic wave transmitted from each of the ultrasound elements that constitute the probe 410, in such a manner that the ultrasonic waves converge at a predetermined depth, so as to generate transmission beams that converge at the predetermined depth.

The D/A converter 430 converts electrical signals of transmission pulses from the transmit beamformer 420, into acoustic signals. The A/D converter 440 converts the acoustic signals received by the probe 410, being reflected in the process of propagation within the examination target 101, into electrical signals again, to generate receive signals.

The beamformer memory 450 stores via the A/D converter 440, in every transmission, beamforming delay data as to each focused point of the receive signals outputted from the ultrasonic elements. The receive beamformer 460 receives via the A/D converter 440 in every transmission, the receive signals outputted from the ultrasound elements, and generates beamforming signals from the beamforming delay data as to each transmission stored in the beamformer memory 450, and the receive signals thus received.

The image processor 470 generates an ultrasound image by using the beamforming signals generated by the receive beamformer 460, and automatically extracts an image optimum for measurement, from the 3D volume data being imaged or from a group of 2D cross sectional images accumulated within cine memory. For this purpose, the image processor 470 is provided with a data reconstructing unit 471 configured to generate the ultrasound image by using the beamforming signals generated by the receive beamformer 460, data memory 472 configured to store image data generated by the data reconstructing unit, a model introducer 473 configured to introduce a downsized machine learning model installed on the device in advance, a cross section extractor 474 configured to use the machine learning model to automatically extract an image optimum for measurement from the 3D volume data or from a group of 2D cross sectional images acquired from the data memory 472, an automatic measurement unit 475 configured to perform automatic measurement of a specified region on the cross section thus extracted, and a cross section adjuster 476 configured to receive a user operation input. Though not illustrated, in order to support Doppler imaging, there may be provided a Doppler processor for processing Doppler signals.

Functions of the data reconstructing unit 471 are the same as conventional ultrasound imaging devices, and the data reconstructing unit generates an ultrasound image such as an image in B-mode, in M-mode, or the like.

The model introducer 473 and the cross section extractor 474 implement functions respectively corresponding to the model introducer 250 and the cross section extractor 230 of the first embodiment, and they have the same configurations as shown in the functional block diagram in FIG. 2. In other words, the model introducer 473 is provided with the model storage unit and the model calling unit, and the cross section extractor 474 is provided with the cross section selector 231, the cross section identifier 233, and the identification-result determiner 234. FIG. 2 will be referred to, when deemed appropriate in the following description. The cross section selector 231 reads volume data or a group of 2D cross sectional images of one patient, out of data stored in the data memory 472. Alternatively, data read from the data memory may be video data obtained by imaging 2D cross sections, or an image dynamically updated. The cross section identifier 233 identifies a target group of cross sectional images selected by the cross section selector 231, by using the learning model introduced by the model introducer 473. The identification-result determiner 235 analyzes the identification result of the cross section identifier 233, and determines whether the identification process is finished or not, and determines the next range for selecting a cross section.

The automatic measurement unit 475 may be configured by software incorporating a publicly known automatic measurement algorithm, and perform measurement of the size and others of a predetermined region, from one or more cross sections being extracted. Then, target measured values are calculated based on the information such as the size according to the given algorithm.

The cross section adjuster 476 accepts via the operation input unit 490, user's modification and adjustment on the cross section displayed on the monitor 480, being extracted by the cross section extractor 475, and provides the automatic measurement unit 475 with a command to change the position of the cross section and to perform reprocessing of automatic measurement caused by such change.

The monitor 480 displays the ultrasound image extracted by the image processor 470, together with a measured value and measurement position of the image. The operation input unit 490 comprises an input device for accepting positional adjustment of the cross section extracted by a user input, switching of the cross section, and adjustment of the measurement position. The image processor 470 performs a part of the processing once again, and updates the display result on the monitor 480.

Next, there will be described a learning model stored in the model storage unit 251 of the model introducer 473.

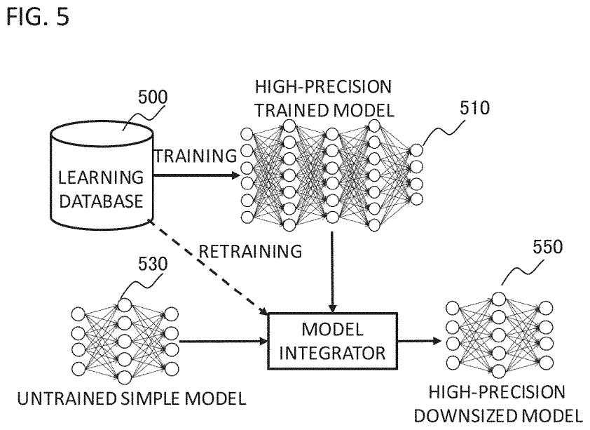

This learning model is a high-precision downsized model installed on the device in advance. As shown in FIG. 5, the downsized model is a simple model 550 installable on the device with keeping precision, being acquired according to a model integrator that integrates an untrained model 530 with a high-precision model 510 having been trained by machine learning with the use of learning database 500. An image processor, a CPU, and others, separate from the ultrasound imaging device 40, can implement functions of the model integrator. If the ultrasound imaging device 40 is equipped with the CPU, this CPU within the device may implement the functions. The learning database 500 stores in advance a large number of image data, for example, 3D fetal images at each week of development, and cross sectional images used for measurement.

A specific structure of the downsized machine learning model will be described, taking as an example, CNN (Convolutional Neural Network) being one type of Deep Learning (DL).

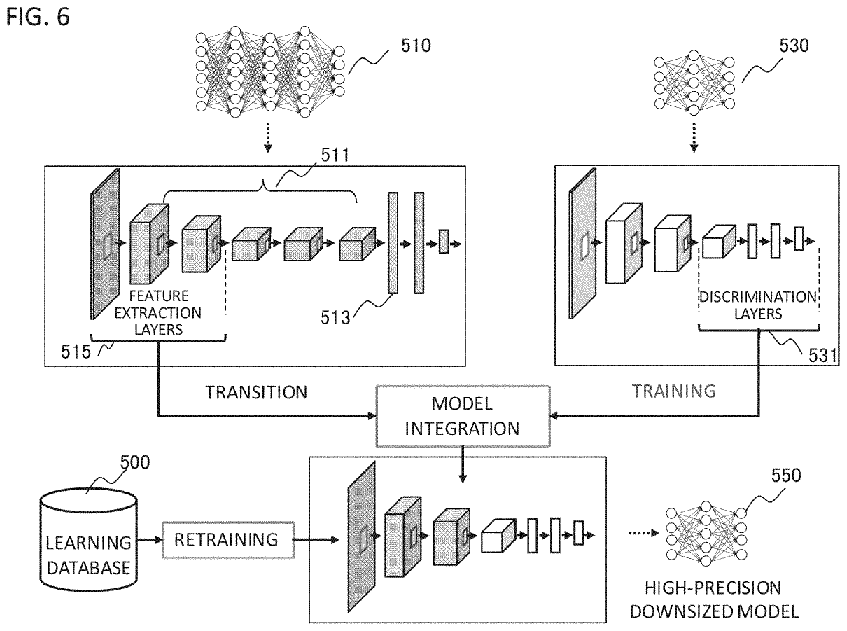

As shown in FIG. 6, in order to ensure high precision, the high-precision trained model 510 has a deep layer structure, provided with a plurality of convolutional layers 511 for extracting a feature amount on the forward stage of the layers. On the backward stage of the layers, there are provided some full connection layers (pooling layers) 513 in a higher dimension for calculating a discrimination score of the feature amount. Among the convolutional layers 511, one or more layers adjacent to the input layer, in particular, contribute to feature extraction, and they are referred to as feature extraction layers 515. Layers in proximity to the full connection layers 513 contribute to discrimination, and they are referred to as discrimination layers. The model 510 has high precision in discrimination, but since the model size is large, long processing time is needed. On the other hand, though the untrained model 530 has a plurality of convolutional layers and full connection layers similar to the model 510, the layer structure is simple and small in size. For example, the number of the convolutional layers is less than the learning model 510, and the number of dimensions of the full connection layers is small. The untrained model 530 is high in discrimination speed, but relatively low in precision.

The downsized model 550 is established by integrating the feature extraction layer 515 as a part of the layer configuration of the trained model 510, with the discrimination layer 531 of the untrained model 530, to structure a new layer configuration, and then retrained using the learning database 500. It is to be noted that the layer configurations of the models, 510, 530, and 550 as shown in FIG. 5, are examples for describing the method of model downsizing. Therefore, the layer configurations are not limited to those as illustrated, but include various layer configurations usable for the aforementioned downsizing method.

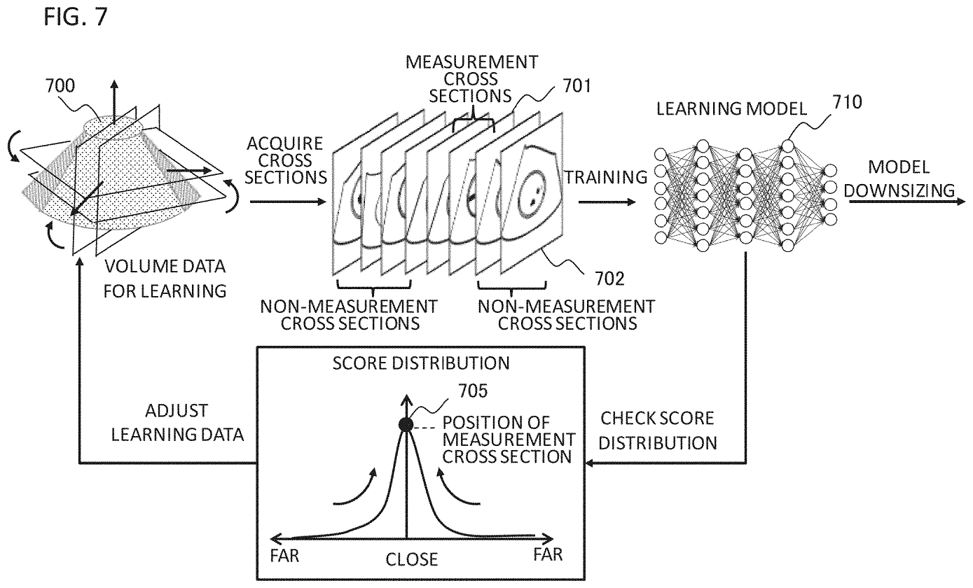

Next, with reference to FIG. 7, a method for creating the trained model 510 (training process) will be described. FIG. 7 illustrates how to create the learning model to implement high-speed and high-precision search. As shown in FIG. 7, a group of measurement cross sections 701 and a group of non-measurement cross sections 702 (cross sections that are not the measurement cross sections) are generated from volume data for learning 700, and machine learning is performed using those cross sections as learning data. Then, there is obtained a learning model 710 for automatically extracting features of the measurement cross sections and features of the non-measurement cross sections. The learning model calculates as to each inputted cross section (cross section for discrimination), a score (referred to as “discrimination score”) representing to what degree the cross section includes features of the measurement cross section. Then, a distribution of the scores (score distribution) 705 is generated, plotting the scores calculated for the plurality of cross sections, respectively. In the figure, there is shown a simplified distribution being expanded one dimensionally, but in actual, this distribution can be shown three-dimensionally. Typically, in volume data of a living body, spatially closer to the position of the measurement cross section indicates that the cross section has a higher discrimination score. Therefore, as shown in FIG. 7, the score distribution 705 should have a form showing the score is the highest at the center, when the position of the measurement cross section is provided as the center, and the score becomes lower, as the cross section goes away from the center.

In the process of training the learning model, the score distribution 705 as an output from the learning model is checked to obtain the distribution where the discrimination score of a cross section becomes higher, as the cross section becomes spatially closer to the position of the measurement cross section. In order to achieve this distribution, machine learning is repeated while adjusting weighting factors of the layers constituting the model, together with adjusting the learning data. In adjusting the learning data, anatomical information of a living body is used to adjust the spatial distance between the non-measurement cross section and the measurement cross section, and the position where the cross sections are acquired. According to such iteration of the adjustment as described above, a high-precision learning model that is suitable for searching for the measurement cross section can be generated, on the basis of the distribution of discrimination scores. In the case where there is a plurality of measurement cross sections, as a processing target, the learning model is created for each of the plurality of measurement cross sections.

When the learning data is not volume data, but temporally sequential 2D cross sections, the horizontal axis of the score distribution 705 in FIG. 7 is changed from spatial axis to temporal axis. Then, the cross sections within the frame close to the measurement cross section are found to be similar to the measurement cross section. Using this result, the sampling intervals of the learning data are adjusted so that the discrimination score becomes higher as the cross section is positioned closer to the measurement cross section in temporal axis. Accordingly, the learning model can be created in a similar manner that uses volume data as the learning data.

The aforementioned downsized model 550 is also trained in the same manner as described above, the downsized model being obtained by integrating thus trained model 510 with untrained model 530. In the time of retraining, the learning rate of the trained model 510 and the untrained model 530 is adjusted so that the learning is performed emphasizing the discrimination layer 531. In other words, the weighting factor of the feature extraction layer 515 moved from the trained model 510 is maintained, and the learning rate of the discrimination layer 531 moved from the untrained model 530 is raised. Then, this allows acquisition of the downsized model 500 achieving both high precision and high-speed processing.

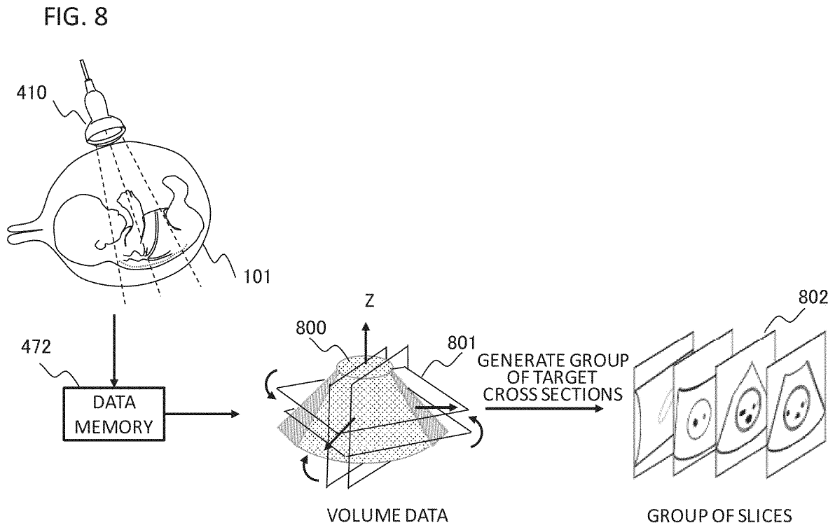

In light of the aforementioned configuration of the ultrasound imaging device 40, there will be described a process for extracting a cross section optimum for measurement, according to each unit of the cross section extractor 474 of the present embodiment. As one example, there will be described a case where the biparietal diameter (BPD), abdominal circumference (AC), and femur length (FL) of an unborn baby (fetus) are measured to estimate the weight. As shown in FIG. 8, in estimating the fetal weight, volume scanning is performed on the fetus 101 being an examination target, by using a mechanical probe or an electronic 2D probe 410, and volume data is stored in the data memory 472. The cross section extractor 474 calls thus acquired volume data 800 from the data memory 472, and cross sections are cut out at cut positions 801 within the search area thus determined. Then, a group of target cross sections 802 are acquired. The cross sections being cut out include a plane perpendicular to the axis (Z-axis) of the volume data, a plane parallel to the Z-axis, and a plane rotated in the deflection angle direction or in the elevation angle direction.

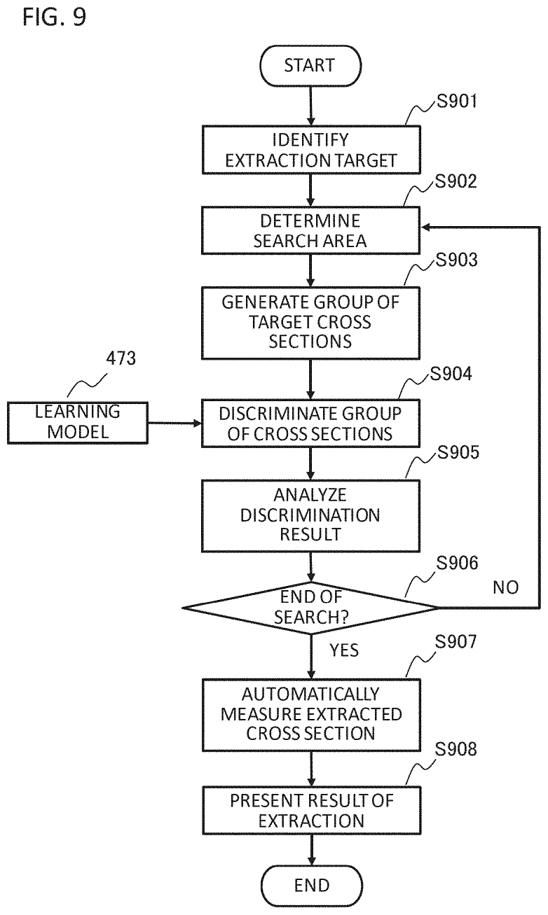

With reference to FIG. 9, a specific example of the processing steps for the cross section extraction will be described. A user's instruction to start the extraction triggers the processing of cross section extraction. An instruction to start measurement may function as the instruction to start the extraction.

When the processing of cross section extraction starts, the cross section extractor 474 (FIG. 2: cross section selector 231) initially reads out from the data memory 472, volume data or sequentially scanned 2D-image group of one patient specified in advance by an operator, and identifies an input format, a type of extraction target, and a type of cross section to be extracted, for the data targeted for processing (step S901). For example, identification of the input format indicates to determine whether the input is 3D data or 2D data. The type of extraction target and the type of cross section is identified responding to the purpose of the measurement when there are a plurality of regions and cross section types to be extracted.

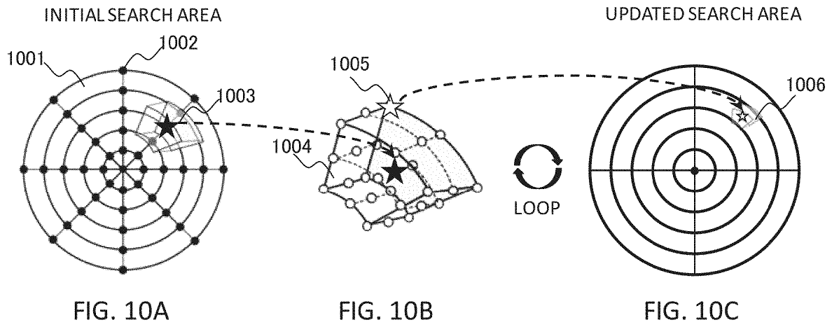

The process in step S902 is performed according to the “coarse to fine approach” that sequentially narrows down an area targeted for extracting a cross section (search area) starting from a large area. Therefore, the cross section selector (FIG. 2: 231) firstly determines an initial search area (step S902), and generates a group of target cross sections (step 903). FIG. 10 shows one example for determining the search area according to the coarse to fine approach. FIGS. 10(a) and (c) are plan views showing the volume data schematically provided about the rotation axis, the volume data being a solid of revolution of fan-shaped plane. As shown in FIG. 10(a), the initial search area 1001 includes the whole area of the volume data, and sampling points (black points) 1002 are provided at relatively coarse intervals in the deflection angle direction and in the radial direction. Then, there are extracted cross sections positioned in the direction of tangential line of the solid of revolution that passes through the sampling point 1002.

Next, the cross section identifier (FIG. 2: 232) applies to thus extracted group of cross sections, the learning model (FIG. 6: downsized learning model 550) called in advance from the model introducer 473, discriminates each of the cross sections of the cross section groups, and acquires scores representing the proximity of the cross sections to the target cross section (step S904). Processing according to the learning model 550 can be performed in parallel on individual cross sections of the cross section group, and a score distribution can be obtained as a totaled result of the scores of individual cross sections. The learning model used in step S904 is created through the learning process as shown in FIG. 7, for each type of the measurement cross sections; BPD measurement cross section, AC measurement cross section, and FL measurement cross section. Those created learning models are stored in the model storage unit (251), and the model calling unit (252) introduces the learning model associated with the measurement cross section that is a processing target.

The cross section extractor 474 analyzes the score distribution as a result of discrimination of each cross section according to the learning model (step S905) and narrows the initial search area 1001 down to a smaller search area. As shown in FIG. 7, in the score distribution, the horizontal axis represents the distance from the target cross section, and the vertical axis represents the scores, and the next search area is narrowed down to an area that is close to a peak. If there is a plurality of peaks, the search area is determined in a manner that includes the plurality of peaks. In the example as shown in FIG. 10(b), the center 1003 of the next search area and the search range 1004 are determined as a result of step S905, and a group of cross sections (cross sections including sampling points indicated by white circles) are extracted. The learning model is applied to this group of cross sections, similarly, and the score distribution is acquired. Then, the area is further narrowed down for extracting the group of cross sections.

As described above, in step S905, it is determined whether the search area is narrowed sufficiently on the basis of the analysis result of the score distribution, and across section suitable for the measurement is found. Then, it is further determined whether the search is to be finished (step S906). If the search is not finished, a new search area is determined, approaching a region that seems to include the measurement cross section, on the basis of the analysis of the result (step S902).

The processing from step S902 to step S906 is repeated two or more times, and along with narrowing the search area, an optimum measurement cross section is extracted, enabling a complete search at high speed. At the time when the search area becomes small to a certain degree, the direction (angle) of the cross section may be changed not only in the deflection angle direction but also in the elevation angle direction. As described above, narrowing the search area is repeated two or more times like a loop, thereby enabling extraction of the measurement cross section having a high score, with less number of identification processes.

When it is determined that the search is finished in step S906, automatic measurement or manual measurement as appropriate is performed on thus extracted measurement cross section (step S907). Finally, there are presented a plurality of extraction results, such as the extracted cross section, information of the cross section in the space, a measured value and measurement position, and other higher-ranked candidates (step S908). The monitor 480 displays thus presented extraction results and the processing is finished.

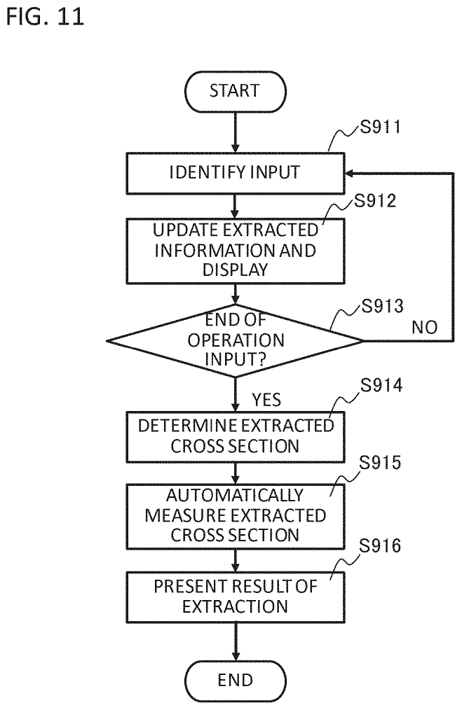

The automatic extraction of the cross section is a subsidiary diagnostic function, and it is necessary for a user to determine a final diagnosis. In the present embodiment, the cross section adjuster 476 accepts a signal from the operation input unit 490, and this allows adjustment of the cross section, switching of the cross section, and re-evaluation of measurement according user preference with a simple operation. FIG. 11 shows the process of the cross section adjustment. The cross section adjustment starts upon receipt of a signal from the operation input unit 490 that accepts user's screen operation, after completion of aforementioned extraction and displaying of the measurement cross section. In response to the input signal, the type of input operation is identified to know which instruction is given; adjustment of cross section, switching of the cross section, or re-evaluation of measurement (step S911). In response to the input signal, details of the screen display and information of the cross section held inside are updated in real time (step S912). Then, it is determined whether the operation input is to be finished (step S913). At the end of the operation input, a finally extracted cross section is determined (step S914). Thereafter, similar to the process as shown in FIG. 9, there are performed processing steps such as automatic measurement on the adjusted cross section (step S915), presenting information including the extracted cross section and measured results (step S916), and displaying the information on the monitor 480.

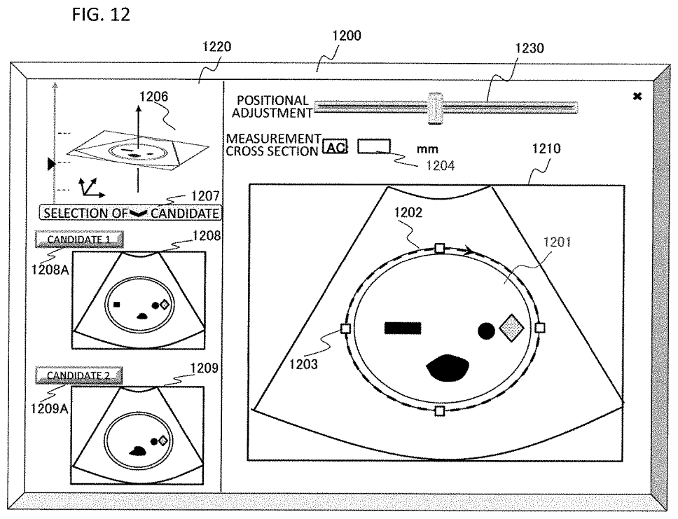

FIG. 12 shows one example of the screen (UI) displayed on the monitor 480. This figure illustrates an example of the AC measurement cross section, and blocks are displayed on the display screen 1200, such as a block for displaying the measurement cross section 1210, a block for displaying cross section candidates 1220, a slider for positional adjustment 1230, and a block showing a type of the cross section and measured value. The measurement cross section 1201 extracted by the cross section extractor 474 is displayed in the block for displaying the measurement cross section 1210. Further, there are displayed the position 1202 and the measured value 1204 obtained from measurement performed on the measurement cross section 1201. A marker 1203 draggable by user's manipulation is displayed on the measurement position 1202. By dragging the marker 1203, the measurement position 1202 and the measured value 1204 are updated.

In the block for displaying cross section candidates 1220, there may also be displayed a spatial positional relationship 1206 of each cross sectional image in 3D volume data, together with an UI (candidate selection field 1207) for selecting a candidate. When the user requests to change the extracted measurement cross section, the candidate selection field 1207 is expanded and non extracted candidate cross sections 1208 and 1209 are displayed. The candidate cross sections may include, for example, a cross section positioned close to the extracted cross section, or a cross section with a high score, and in the figure, there are displayed two candidates. However, the number of candidates may be three or more. There may also be provided buttons 1208A and 1209A prompting to select any of the candidate cross sections.

The slider for positional adjustment 1230 is a UI for adjusting the position, enabling selection of a cross sectional image from any position on the volume data, for instance. When the user manipulates the slider for positional adjustment or the candidate buttons 1208A, 1209B, and others, the operation input unit 490 transmits a signal to the cross section adjuster 476, in response to the user's manipulation. The cross section adjuster 476 performs a series of processing such as updating and switching of the cross section, updating the measurement position, and updating of the measured value, and then, displays a result of the processing on the monitor 480.

When there is a plurality of cross sections targeted for measurement, the procedures shown in FIG. 9 and FIG. 11 are repeated as to each cross section, and then results of the measurement are obtained. For the example as described above, the measurement result is obtained as to each of the BPD measurement cross section, the AC measurement cross section, and the FL measurement cross section.

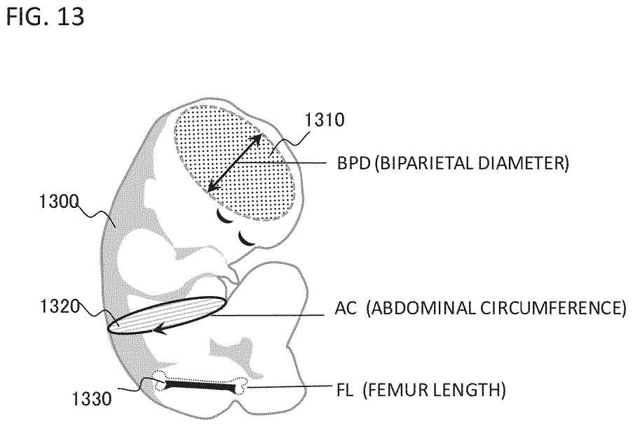

The automatic measurement will be described specifically, taking fetal weight measurement as an example. As illustrated in FIG. 13, the fetal weight measurement is performed on a fetal structure 1300 being a measurement target. That is, BPD (biparietal diameter) is measured from the fetal head cross section 1310, AC (abdominal circumference) is measured from the abdominal cross section 1320, and FL (femur length) is measured from the femur cross section 1330. Then, the fetal weight is estimated on the basis of those measured values, and it is determined whether the fetus is growing without any problems, according to comparison with a growth curve in association with the number of weeks.

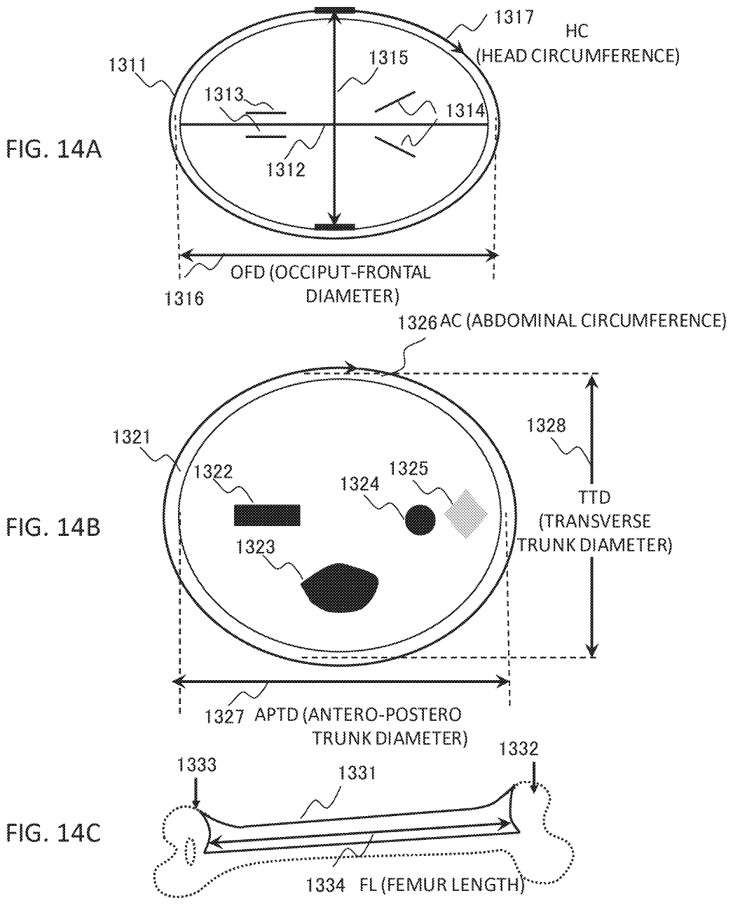

As illustrated in FIG. 14 (a), as for the fetal head cross section, a cross section with structural features, such as the skull 1311, medium line 1312, septum pellucidum 1313, and quadrigeminal cistern 1314, is recommended as the measurement cross section, according to guidelines. The measurement target may be different depending on countries. For example, in Japan, BPD (biparietal diameter) 1315 is measured from the fetal head cross section, whereas in Western countries, typically, OFD (occiput-frontal diameter) 1316 and HC (head circumference) 1317 are measured. The measurement position as a target may be provided in prior settings of a device, or provided before performing the measurement. The measurement may be performed by the automatic measurement unit 475 (FIG. 4), for example, according to an automatic measurement technique such as the method as described in Patent Literature 1. In this technique, for the case of a head part, an oval shape corresponding to the head part is calculated based on features of a tomographic image to obtain the diameter of the head part.

As shown in FIG. 14(b), as for the fetal abdominal cross section, across section having structural features such as an abdominal wall 1321, a umbilical vein 1322, a stomach vesicle 1323, an abdominal aorta 1324, and a spine 1325, is recommended as the measurement cross section, according to guidelines. Typically, AC (abdominal circumference) 1326 is measured. Depending on locale, APTD (antero-postero trunk diameter) 1327 and TTD (transverse trunk diameter) 1328 may be measured. The measurement position as a target may be provided in prior settings of a device, or provided before performing the measurement. The measurement method may be the same as the case of measuring the head part.

As shown in FIG. 14(c), as for the fetal femur cross section, a cross section having structural features such as the femur 1331, distal ends 1332 being both ends of the femur, and proximal ends 1333, is recommended as the measurement cross section, according to guidelines. From this measurement cross section, FL (femur length) 1334 can be measured. The automatic measurement unit 475 calculates an estimated weight, using each of the values (BPD, AC, and FL) measured at the three cross sections, according to the following formula, for example:

Estimated weight=a×(BPD)³+b×(AC)²×(FL)

(where a and b are factors obtained based on empirical values, for example, a=1.07, b=0.30) The automatic measurement unit 475 displays thus calculated estimated weight on the monitor 480.

Embodiments of the ultrasound imaging device have been described, taking as an example, extraction of cross sections necessary for measuring fetal weight, including the AC measurement cross section, the BPD measurement cross section, and FL measurement cross section. The present embodiments features that identification and extraction on the basis of the downsized learning model, and it is further applicable to extraction of 4CV cross section of heart (heart four chamber view) for checking fetal cardiac function, 3VV cross section (three vessel view), left ventricular outflow view, right ventricular outflow view, and aortic arch view, and also applicable to automatic extraction of measurement cross section of amniotic fluid pocket for measuring the amount of amniotic fluid surrounding the fetus. In addition, the embodiments above may be applicable to automatic extraction of a standard cross section necessary for measurement and observation of heart and circulatory organs, not only in fetus but also in adults.

According to the present embodiments, a highly sophisticated learning model is employed, enabling automatic and high-speed cross section extraction, though the cross section extraction is highly operator dependent. Using the downsized model, obtained by integrating the learning model having a highly trained layer configuration, with the learning model having a relatively simple layer configuration, facilitates implementation of the learning model in the ultrasound imaging device, and enables high-speed processing.

According to the present embodiments, the coarse to fine approach is employed in extracting the cross section, and this enables a high-speed and complete search for the cross section.

### Modification of Second Embodiment

In the aforementioned embodiments, there has been described the case where the volume data imaged in one-time examination for one patient is processed. The present embodiment is applicable to a group of 2D images taken in the examination at a previous time or in the examinations across the past several times. There will now be described the case where input data is 2D images that are temporally sequential.

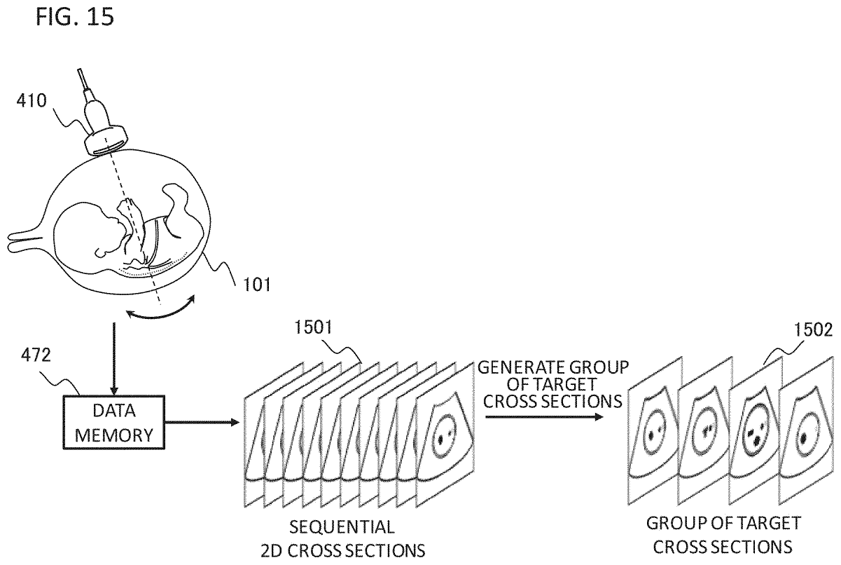

FIG. 15 illustrates data acquisition and generation of a group of cross sections from data memory, when an extraction target is sequential 2D cross sections on temporal axis. In the present embodiment, a 1D probe is moved on the fetus 101 being an examination target, and temporally sequential 2D cross sections are accumulated in the data memory 472. Sampling of the cross section data 1501 called from the data memory 472 is performed on the temporal axis, and a target group of cross sections 1502 are generated. In other words, the search area on the temporal axis is determined, thereby selecting a frame image on the temporal axis. In determining the search area, the coarse to fine approach may be employed as in the case of volume data described above.

Thereafter, the cross section identifier (233) identifies the target group of cross sections according to the learning model called from the model introducer 473 in advance. A distribution on the temporal axis as a result of the identification is analyzed, the search is finished when a cross section suitable for the measurement is found, and a measurement cross section is determined. If imaging is performed continuously in parallel to this image processing, the cross section called from the data memory may be updated according to imaging manipulation by a user at the point of time.

In FIG. 15, there has been described the case where the 2D cross sections are called from the data memory 472. The read out data may be 3D volume data acquired by one-time scanning, or a plurality of 3D volume data obtained by sequentially scanned in 4D mode. When the input data corresponds to a plurality of 3D volume data, one cross section is extracted from one volume data, then the volume is changed and a cross section thereof is extracted.

Finally, one cross section is determined from the candidate cross sections extracted from the plurality of volume data.

### Other Modifications

In the second embodiment and its modification, the present invention is applied to the ultrasound imaging device, but the present invention may also be applicable to any medical imaging device that is capable of acquiring volume data or time-series data. In the aforementioned embodiments, there has been described the case where the image processor is a constitutional element of the medical imaging device. However, if imaging and image processing are not performed in parallel, the image processing of the present invention may be performed in an image processing device or an image processor that are spatially or temporally away from the medical imaging device (the imager 100 in FIG. 1).

In addition, the embodiments and modifications of the present invention are described in detail for ease of understanding, and those embodiments and modifications are not necessarily limited to those as described above including all the components. A part of or all of the configurations, functions, processors, and processing means described in some of the above embodiments may be implemented by hardware, for example, by designing with an integrated circuit. Those configurations, functions, and others may be implemented by software, by interpreting and executing programs for processors to implement each of the functions. Information such as programs, tables, and files for implementing each of the functions may be placed in storage such as memory, hard disk, and SSD (Solid State Drive), or in a storage medium such as IC card, SD card, and DVD.

### DESCRIPTION OF SYMBOLS

10 medical imaging device
40 ultrasound imaging device
100 imager
101 examination target
200 image processor
230 cross section extractor
231 cross section selector
233 cross section identifier
235 identification-result determiner
250 model introducer
251 model storage unit
253 model calling unit
300 user interface
310 monitor
330 operation input unit
350 memory unit
410 probe
420 transmit beamformer
430 D/A converter
440 A/D converter
450 beamformer memory
460 receive beamformer
470 image processor
471 data reconstructing unit
472 data memory
473 model introducer
474 cross section extractor
475 automatic measurement unit
476 cross section adjuster
480 monitor
490 operation input unit
500 learning database
510 high-precision trained model
530 simple untrained model
550 high-precision downsized model

What is claimed is:

1. A medical imaging device comprising,
an imager configured to collect image data of a subject, and
an image processor configured to extract a specified cross section from the image data collected by the imager, wherein,
the image processor comprises,
a model introducer configured to introduce a learning model being downsized by integrating a feature extraction layer of a trained model with a discrimination layer of an untrained model, and being trained in advance to output discrimination scores for a plurality of cross sectional image data, the discrimination score representing spatial or temporal proximity to the specified cross section, and
a cross section extractor configured to select a plurality of cross sectional images from the image data and to extract the specified cross section on the basis of a result of applying the learning model to the cross sectional images being selected.

2. The medical imaging device according to claim 1, wherein,
the model introducer comprises,
a model storage unit configured to store a plurality of learning models prepared in response to types of the cross section to be extracted, and
a model calling unit configured to call learning models associated with the plurality of cross sectional images selected by the cross section extractor, out of the plurality of learning models, and to pass the learning models to the cross section extractor.

3. The medical imaging device according to claim 1, wherein,
the cross section extractor comprises,
a cross section selector configured to select a plurality of cross sections from the image data collected by the imager,
a cross section identifier configured to apply the learning models to the cross sections selected by the cross section selector, and
an identification-result determiner configured to determine a result of the cross section identifier.

4. The medical imaging device according to claim 3, wherein,
the cross section extractor repeats processing of the cross section selector and the cross section identifier, in response to the result from the identification-result determiner, and
the cross section selector changes or narrows down an area of the image data targeted for selecting the plurality of cross sections, at each iteration.

5. The medical imaging device according to claim 1, further comprising a cross section adjuster configured to accept adjustment according to a user on the cross section being extracted, wherein,
the cross section extractor reruns a part of processing, in response to an instruction of the adjustment accepted by the cross section adjuster.

6. The medical imaging device according to claim 5, further comprising a monitor configured to display a result the processing of the cross section extractor, wherein,
the monitor updates displayed details, when the cross section extractor reruns the processing.

7. The medical imaging device according to claim 1, wherein,
the image data collected by the imager is three-dimensional volume data.

8. The medical imaging device according to claim 1, wherein,
the image data collected by the imager is time-series image data.

9. The medical imaging device according to claim 1, wherein,
the imager is an ultrasound imager comprising,
a probe configured to transmit and receive ultrasound signals, and
an image generator configured to generate an ultrasound image by using the ultrasound signals received by the probe.

10. An image processing method for determining from imaged data, a target cross section to be processed and for presenting the target cross section, comprising,
preparing a learning model that is trained in advance to output discrimination scores for a plurality of cross sectional images, the discrimination score representing spatial or temporal proximity to an image of the target cross section, and
obtaining by using the learning model, a distribution of the discrimination scores of the plurality of cross sectional images selected from the imaged data and determining the target cross section on the basis of the distribution, wherein,
the learning model is a downsized model obtained by integrating a feature extraction layer of a trained model that is trained in advance by using as learning data, a plurality of cross sectional images and the image of the target cross section constituting the imaged data, with a discrimination layer of an untrained model, followed by retraining.

11. The image processing method according to claim 10, wherein,
determining the target cross section repeats, selecting a plurality of cross sections from a specified area of the imaged data and obtaining a distribution of the discrimination scores of the plurality of cross sections being selected, and
narrowing down the area for selecting the plurality of cross sections at each iteration.

12. The image processing method according to claim 10, wherein,
the imaged data is three-dimensional volume data or time-series image data acquired by an ultrasound imaging device.
